(19) United States
(12) Patent Application Publication
(10) Pub. No.: US 2012/0083881 A1
(43) Pub. Date: Apr. 5, 2012

(54) Stand-Alone Anterior Fusion Device

(76) Inventor: Roderick Claybrooks, Franklin, MI (US)

(21) Appl. No.: 12/896,850

(22) Filed: Oct. 2, 2010

(51) Int. Cl. A61F 2/44; A61B 17/88

(52) U.S. Cl. 623/17.11; 606/279

(57) ABSTRACT

Disclosed herein is a stand-alone anterior fusion device that is positioned in the space between adjacent vertebrae to be fused. The device comprises an annular cage having a thickness defined by an outer surface and an inner surface. The inner surface is defined by a cavity adapted to receive graft material. A top surface is in a spatial relationship to a bottom surface and may be further defined by a Lordosis angle. Ascending and descending guides extend through the wall, each guide has an axis defined by an attitude, where each guide directs a vertebral screw in a desired trajectory into the adjacent vertebrae whereby a bi-cortical screw purchase may be achieved. The device is a stand alone stabilizing device as well as a cage for containing graft material and maintaining the height of the space between the vertebrae to be fused.

### FIELD OF THE INVENTION

The present invention relates to a device and method for spinal fusion. More specifically, the present invention relates to a stand-alone device for spinal fusion that is positioned between the vertebrae to be fused and a method for stabilizing adjacent vertebrae through an anterior approach by inserting the stand-alone device between the vertebrae to be fused.

### BACKGROUND OF THE INVENTION

The human spine typically comprises 33 vertebrae. Seven cervical, twelve thoracic, and five lumbar vertebrae comprise the movable presacral section of the spine. Five fused elements form the inflexible sacrum that articulates with the pelvis. Caudal to the sacrum, four or five ossicles make up the coccyx. A typical vertebra consists of two essential parts: an anterior segment, the vertebral body, and a posterior part, the vertebral or neural arch. The vertebral arch consists of a pair of pedicles and a pair of laminae, and supports seven processes: four articular, two transverse, and one spinous process. The laminae, pedicles, and processes enclose two lateral foramen, and the central spinal canal. The spinal cord exits the brain in the skull and enters the spine through the spinal canal. The branching nerves exit the spine through the lateral foramen.

The vertebral body is generally oval shaped from a top view and has a hard, strong cortical rim around the outside containing a soft cancellous bone on the inside. There are twenty three intervertebral discs which are fibrocartilaginous cushions that act as shock absorbers. These intervertebral discs are comprised of the annulus, a tough fibrous outer layer, and the nucleus, a soft gelatin like inner layer. The intervertebral discs provide six degrees of freedom: axial translation, axial rotation, lateral translation, lateral rotation, forward and back translation and forward and back rotation. This freedom allows of flexion and extension, left and right side bending and left and right rotation. Throughout this movement, when the spine is operating properly, the nerves are maintained clear of the hard structure of the spine.

The spinal cord passes through the spinal canal to allow nerves from the brain to pass to various portions of the body. Spinal nerve roots emerge from the spine at different locations along the length of the spine. In a healthy spine, the spinal cord and nerve roots are protected from damage by the structure of the spine. Over time, or because of accidents, the intervertebral discs may loose height, become cracked, dehydrated, or herniated. The result is that the disc height is reduced leading to compression of the spinal cord or nerve roots, causing pain and in some cases damage to the nerves. For example, when a herniated disc occurs, a small portion of the nucleus pushes out through a tear in the annulus into the spinal canal. This can irritate a nerve and result in pain, numbness or weakness in the back, legs or arms.

When there is a problem with an intervertebral disc, such as a herniated disc, and the physician determines spinal fusion is the best course of action, a surgeon removes the intervertebral disc between two adjacent vertebrae. The surgeon must be careful not to damage a nerve in the process. The surgeon also prepares the site to accept bone grafts. These specially prepared bone fragments come either from the patient (autograft), usually the hip, or a donor (allograft) and are packed in the space between the adjacent vertebrae formerly occupied by the intervertebral disc. To preserve, or restore, the height of the space between the vertebrae, a cage or bone graft is placed in the disc space. Preserving the space between the adjacent vertebrae maintains the location where spinal nerve roots emerge from the spinal cord.

One type of spinal fusion procedure used to treat problems such as disc degeneration, disc herniation, and spine instability is a posterior lumbar interbody fusion (PLIF). In this procedure, the surgeon works on the spine from the back (the posterior) and removes a spinal disc in the lumbar region. The surgeon inserts bone graft material into the interbody space between the two vertebrae where the disc was removed. The graft may be held in place with a fusion cage. The goal of the procedure is to fuse the vertebrae by stimulating the two adjacent vertebrae to grow together into one solid bone. The fusion procedure creates a rigid and immovable column of bone joining the two adjacent vertebrae that were formerly separated by the damaged intervertebral disc.

Traditionally, to support and stabilize the vertebrae, one or more rods or braces are attached to the vertebrae to be fused with the purpose of the rods being to support the vertebrae, usually along the posterior of the spine while fusion takes place. These rods are typically attached to the vertebrae by anchors which are fitted into the pedicle of the vertebrae, typically by employing a pedicle screw. Pedicle screws come in a variety of lengths, diameters, and thread types. These rods are often held in place by anchors which are fitted into the pedicle screw.

The benefits of a bi-cortical purchase (screw engagement of two cortical bone sections) over unicortical purchase (screw engagement of one cortical bone section) are well documented. Engagement of two layers of cortex provides greater vertebral screw stability regarding pull out strength and wobble. Therefore, in an anterior approach, bi-cortical purchase by the vertebral screw is preferred.

The art is replete with different types of cages for spinal fusion. One example of a cage is disclosed in U.S. Pat. No. 7,771,473. The '473 patent discloses an expandable spinal fusion device. Although the cage of the '473 patent is adjustable, claiming to reduce trauma to the surrounding tissue, there appears to be no means to stabilize the superior and inferior vertebrae through the use of the disclosed cage. As a result, additional hardware will be needed to stabilize the adjacent vertebrae. The device of the '473 patent requires a second, posterior approach surgery to reinforce and stabilize the vertebrae to be fused.

One solution for a stand alone device is found in U.S. Patent Application No. 20020032483. The 20020032483 Application discloses a fusion device that replaces the intervertebral disc and provides cavities for allograft material. In one embodiment, the device is fastened to adjacent vertebrae by a dovetail joining method where a jig is used to cut the adjacent vertebrae to form a slot to receive a dovetail formed in the interbody fusion device. An immediate concern is the difficulty in preparing the adjacent vertebrae and the proximity to the spinal cord. Additionally, should the procedure be ineffective, the surface condition of the modified vertebrae is a concern, as a significant about of cortical material would be removed in the process. Additionally, there is nothing preventing anteroposterior movement of the device relative to the vertebrae.

The step of cutting bone material to provide a fusion device was obviated in U.S. Patent Application No. 20080154379. The 20080154379 Application discloses a fusion device with cortical cap members having slots formed therein for receiving a body member having dovetail projections. The device eliminates concerns associated with cutting and shaping the vertebra of the device disclosed in the 20020032483 application, however, no means of attaching the fusion device to the adjacent vertebrae is disclosed.

There are several advantages to an anterior approach over a posterior approach in a spinal fusion procedure. More of the intervertebral disc may be removed by an anterior approach, which is by making an incision in the abdomen to access the spine, than through a posterior approach, accessing the spine through the back. When a surgeon performs a discectomy from the posterior, the spinal cord must be protected and the back muscle is cut. An additional benefit of the anterior approach is the surgeon is further from the spinal cord and nerve roots. It would be desirable to avoid a posterior approach.

The deficiencies of the market are that currently a second procedure is required to stabilize the adjacent vertebrae if an anterior approach is used to perform a discectomy. If an anterior device is used, a bi-cortical purchase would be desirous.

It would be desirous to provide a fusion device that is inserted by an anterior approach that also stabilizes the superior and inferior vertebrae without the need for an additional procedure. Therefore, there is a need to provide a stand alone fusion device that can restore the height of the disc space once the intervertebral disc is removed while also stabilizing the adjacent vertebrae. Additionally, if a single device could restore height, contain the graft material and stabilize the adjacent vertebra without additional hardware, it would obviate the need for an additional procedure.

### SUMMARY OF THE INVENTION

A fusion device for providing a desired spatial relationship between adjacent vertebrae comprises an annular cage having a wall with a thickness defined by an outer surface and an inner surface. The inner surface is defined by a cavity within the cage. The cage has a top surface and a bottom surface that are in a spatial relationship to one another. The cage has at least one ascending guide extending through the wall from the outer surface through the top surface and at least one descending guide extending through the wall from the outer surface through the bottom surface. Each guide has an axis defined by an attitude to direct a vertebral screw in a desired trajectory. A vertebral screw is disposed in the ascending guide and descending guide.

The device may comprise a second ascending guide extending through the wall from the outer surface through the top surface and a second descending guide extending through the wall from the outer surface through the bottom surface. The ascending guides and descending guides may be disposed symmetrically with respect to the midsagittal plane of the cage. Alternatively, the ascending guides and descending guides may be disposed asymmetrically with respect to the midsagittal plane of the cage. In still another embodiment, the device may comprise a plurality of ascending guides and a plurality of descending guides.

The top surface may lie within a first plane and the bottom surface may lie within a second plane. Each of the ascending guides may have a different slope with respect to the first plane. Additionally, each of the descending guides may have a different slope with respect to the second plane. To assist in durability and force distribution, each guide may have a seat where the head of a vertebral screw rests or shoulders. Additionally, each seat may be recessed and have a concave contour.

The top surface and bottom surface are preferably denticulated to provide a bite on the superior and inferior vertebrae, respectively. In one embodiment, the outer surface of the cage has a shape that generally follows the contour of the body of a vertebra.

The cage is disposed between a superior vertebra and inferior vertebra, where the top surface of the cage is adjacent to the superior vertebra and the bottom surface of the cage is adjacent to the inferior vertebra. It is preferred that at least one ascending guide is disposed to permit bi-cortical screw purchase of a superior vertebra and at least one descending guide is disposed to permit a bi-cortical screw purchase of an inferior vertebra. The ascending guides may be disposed at oblique angles to permit bi-cortical screw purchase of the superior vertebra and descending guides may be disposed at oblique angles to permit bi-cortical screw purchase of the inferior vertebra.

In one embodiment, at least one ascending guide has a diameter sufficient to provide a change in angulation of a vertebral screw to permit bi-cortical screw purchase of a superior vertebra and at least one descending guide has a diameter sufficient to provide a change in angulation of a vertebral screw to permit bi-cortical screw purchase of an inferior vertebra.

The spatial relationship of the top surface of the cage and the bottom surface of the cage is at least partially defined by a Lordosis angle. A plurality of grooves raidially disposed on the top surface and bottom surface of the cage, the grooves adapted to retain sutures that span the cavity to retain graft material in the cavity.

A method for stabilizing adjacent vertebrae by using an anterior fusion device comprises providing an anterior approach to a spine and providing an anterior fusion device comprising an annular cage having a wall with a thickness defined by an outer surface and an inner surface. The inner surface is defined by a cavity therein. The cage has a top surface and a bottom surface, where the top surface is in a spatial relationship to the bottom surface. The cage has at least one ascending guide extending through the wall from the outer surface through the top surface and at least one descending guide extending through the wall from the outer surface through the bottom surface. The cavity is packed with bone graft and inserting the anterior fusion device into the disc space between adjacent vertebrae. A vertebral screw is inserted into each screw guide, whereby the angle of the screw guide permits bi-cortical screw purchase. The method may further comprise the step of securing the bone graft to the cage with sutures, where the sutures are at least partially retained by grooves formed in the cage.

Further objects, features and advantages of the present invention will become apparent to those skilled in the art from analysis of the following written description.

### BRIEF DESCRIPTION OF THE DRAWINGS

FIG. 1 is a perspective environmental view of the fusion device of the present invention disposed in the disc space between a superior vertebra and a inferior vertebra, showing section line 10-10 cutting through a sagittal plane;

FIG. 2 is a anterior environmental view of the fusion device of the present invention disposed in the disc space between a superior vertebra and a inferior vertebra, showing a section line 9-9 through the midsagittal plane;

FIG. 3A is a lateral environmental view of the fusion device of the present invention disposed in the disc space between a superior vertebra and a inferior vertebra, showing a section cut 8-8 through an oblique plane;

FIG. 3B is a lateral view of the fusion device of the present invention disposed in the disc space between a superior vertebra and a inferior vertebra shown in phantom, revealing various bi-cortical purchase axes and the corresponding angles β and γ in the vertical plane relative to the horizontal plane identified by A-A;

FIG. 4A is a perspective view of the fusion device of the present invention, showing a section cut 4B-4B through an ascending guide and descending guide disposed adjacent to the midsagittal plane of the cage;

FIG. 4B is section cut of 4B-4B of FIG. 4A through the midsagittal plane of the cage, bisecting the ascending guide and descending guide;

FIG. 5A is a top view of the fusion device of FIG. 4A, showing a section cut 5B-5B through an oblique plane of the cage;

FIG. 5B is section cut along 5B-5B of the device of FIG. 5A through an oblique plane to reveal a cross section of ascending guide 45 and descending guide 46;

FIG. 6A is an anterior view of the fusion device of FIG. 4A, revealing six screw guides and six seats, also showing a cross section cut 6B-6B through a horizontal plane of the cage;

FIG. 6B is a is a cross section cut along 6B-6B of the cage of FIG. 6A through a horizontal plane;

FIG. 7 is a lateral view the fusion device of FIG. 4A, revealing a Lordosis angle α;

FIG. 8 is a section cut along 8-8 of the fusion device of FIG. 3 through an oblique plane bisecting cortical screws 21 and 25;

FIG. 9 is a section cut along 9-9 of the fusion device of FIG. 2 through a midsagittal plane bisecting cancellous screws 23 and 24;

FIG. 10 is a section cut along 10-10 of the fusion device of FIG. 1 through an oblique plane bisecting cortical screw 25;

FIG. 11 is a perspective view of the fusion device of the present invention revealing how the device is to be inserted into the disc space S between the superior vertebra and inferior vertebra;

FIG. 12 is a perspective environmental view of the fusion device of the present invention disposed between the superior vertebra and inferior vertebra, showing the direction a cortical screw will be inserted into the cage, as governed by an ascending screw guide;

FIG. 13 is a perspective view of the fusion device of the present invention shown packed with graft material, the graft material being held in place with sutures;

FIG. 14 is a perspective environmental view of an alternate embodiment of the fusion device of the present invention disposed in the disc space between a superior vertebra and a inferior vertebra, showing section line 17-17 cutting through an oblique plane;

FIG. 15 is an anterior environmental view of an alternate embodiment of the fusion device of the present invention disposed in the disc space between a superior vertebra and a inferior vertebra;

FIG. 16 is an lateral environmental view of an alternate embodiment of the fusion device of the present invention disposed in the disc space between a superior vertebra and a inferior vertebra;

FIG. 17 is an oblique sectional cut through the device 101 and vertebral bodies 102, 103 of FIG. 14 to reveal a cross section of ascending cortical screw 122 and descending cortical screw 124 to reveal a change in angulation provided by the guide diameter;

FIG. 18 is a perspective environmental view of an alternate embodiment of the fusion device of the present invention with the superior vertebra removed and a bi-cortical screw purchase of the inferior vertebra;

FIG. 19 is a perspective view of an alternate embodiment of the fusion device of the present invention;

FIG. 20 is a top view of an alternate embodiment of the fusion device of the present invention;

FIG. 21 is a lateral view of an alternate embodiment of the fusion device of the present invention, revealing a Lordosis angle α;

FIG. 22 is an anterior view of an alternate embodiment of the fusion device of the present invention, revealing four screw guides and four seats;

FIG. 23 is a perspective environmental view of another alternate embodiment of the fusion device of the present invention disposed in the disc space between a superior vertebra and a inferior vertebra, showing section line 26-26 cutting through a sagittal plane and 28-28 cutting through an oblique plane;

FIG. 24 is an anterior environmental view of another alternate embodiment of the fusion device of the present invention disposed in the disc space between a superior vertebra and a inferior vertebra;

FIG. 25 is a lateral environmental view of another alternate embodiment of the fusion device of the present invention disposed in the disc space between a superior vertebra and a inferior vertebra showing bi-cortical screw purchase;

FIG. 26 is a sectional cut along 26-26 of the device 201 and vertebral bodies 202, 203 of FIG. 23, through a sagittal plane to reveal a cross section of ascending cortical screw 223;

FIG. 27 is a perspective environmental view of another alternate embodiment of the fusion device of the present invention with the superior vertebra removed and a bi-cortical screw purchase of the inferior vertebra;

FIG. 28 a sectional cut along 28-28 of the device 201 and vertebral bodies 202, 203 of FIG. 23, through an oblique plane to reveal a cross section of ascending cortical screw 224;

FIG. 29 is a perspective view of another alternate embodiment of the fusion device of the present invention;

FIG. 30 is a top view of an another alternate embodiment of the fusion device of the present invention;

FIG. 31 is a lateral view of an another alternate embodiment of the fusion device of the present invention, revealing a Lordosis angle α;

FIG. 32 is an anterior view of another alternate embodiment of the fusion device of the present invention, revealing four screw guides and four seats; and

FIG. 33 is an alternate embodiment of the device shown in FIG. 17 revealing hemispherical seats 352 and 354 and corresponding hemispherical headed ascending cortical screw 322 and hemispherical headed descending cortical screw 324 to reveal a change in angulation provided by the guide diameter.

### DESCRIPTION OF THE PREFERRED EMBODIMENT

The present invention provides a stand-alone fusion device for maintaining the spatial relationship between vertebrae to be fused. The present invention further provides a stand-alone device that stabilizes the vertebrae to be fused without additional hardware. The device is inserted through an anterior approach and provides a cavity for bone graft material. Also provided is a method for stabilizing adjacent vertebrae through an anterior approach by inserting the stand-alone device between the vertebrae to be fused.

As used herein, a midsagittal plane shall refer to a longitudinal plane that divides an object into right and left halves and a sagittal plane shall refer to a longitudinal plane that is parallel to the midsagittal plane.

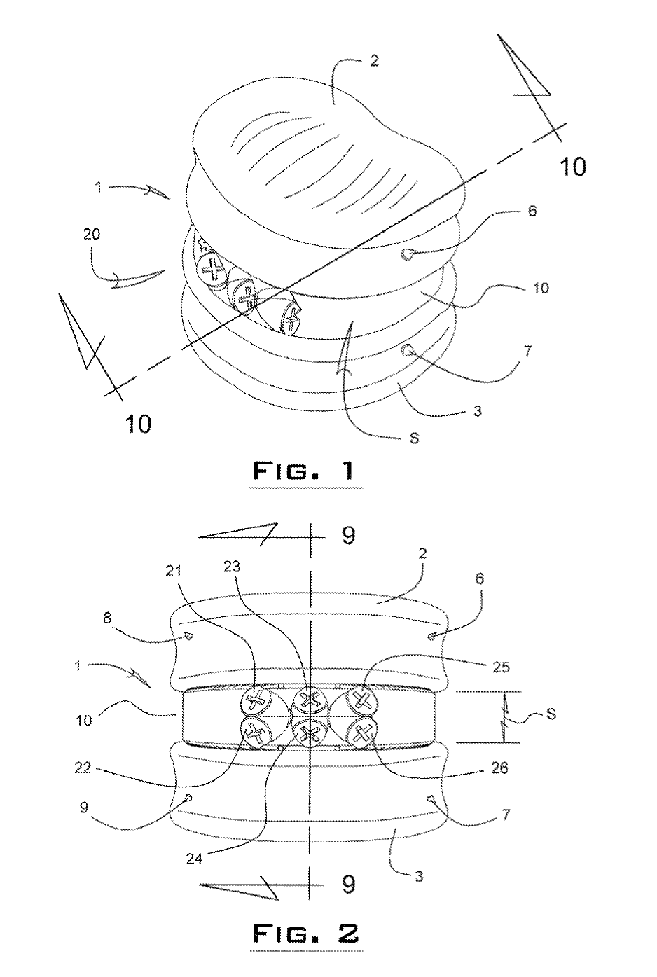

Referring now to FIG. 1, a perspective environmental view of one embodiment of a fusion device 1 according to the principles of the present invention is shown. In the exemplary embodiment, a six screw symmetrically distributed device 1 is disposed between a superior vertebra 2 and an inferior vertebra 3. The embodiment is referred to as symmetrical as the screws are distributed symmetrically about the midsagittal plane. A section line 10-10 cutting through a sagittal plane of the device 1 and superior vertebra 2 and inferior vertebra 3. The device 1 is shown disposed in the disc space S between the superior vertebra 2 and the inferior vertebra 3. The superior vertebra 2, or a vertebra immediately above the fusion device 1, is shown without the pedicles, processes, laminae and spinal canal for clarify of the illustrations herein. Likewise, the inferior vertebral, or a vertebra immediately below the fusion device 1, is shown without the pedicles, processes, laminae and spinal canal for clarify of the illustrations. The vertebrae include an endplate, which is a hard surface at each end of the vertebral body. The end plate is the portion of the vertebra against which the cage 10 rests.

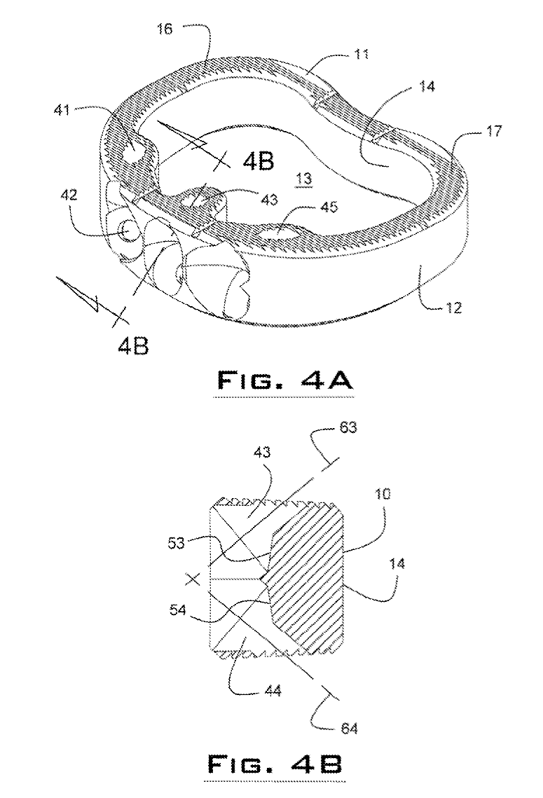

The fusion device 1 provides a desired spatial relationship between the adjacent vertebrae 2, 3. The device 1 comprises an annular cage 10 having a plurality of vertebral screws 20 inserted therein. In the preferred embodiment, at least one screw is directed by a guide to obtain a bi-cortical purchase. As may be seen in FIG. 1, a right superior bi-cortical purchase 6 and right inferior bi-cortical purchase 7 are obtained by vertebral screws 20 of sufficient length and angle, as directed by guides that may be seen in FIGS. 4A-6A. Guides are formed within the cage 10 and direct the vertebral screws toward a desired engagement location of a vertebra. In the preferred embodiment, the cage 10 is formed of polyetheretherketone (PEEK), although those skilled in the art will immediately recognize that other suitable substitutes may be employed in place of PEEK.

Referring now to FIG. 2, an anterior environmental view of the fusion device 1 of the present invention is shown disposed in the disc space S between the superior vertebral body 2 and the inferior vertebral body 3, showing a section line 9-9 through the midsagittal plane, bisecting cancellous vertebral screws 23 and 24. As may be seen more clearly in FIG. 2, the right superior bi-cortical purchase 6 and right inferior bi-cortical purchase 7 are obtained by cortical vertebral screws 25 and 26. A left superior bi-cortical purchase 8 and left inferior bi-cortical purchase 9 are obtained by cortical vertebral screws 21 and 22 respectively, as directed by guides (not shown) formed within the cage 10.

It is contemplated that at least one ascending guide is disposed in the cage 10 to permit bi-cortical purchase of a superior vertebrae and that at least one descending guide is disposed in the cage 10 to permit bi-cortical purchase of an inferior vertebrae. In the preferred embodiment, a vertebral screw is disposed in each ascending guide and descending guide.

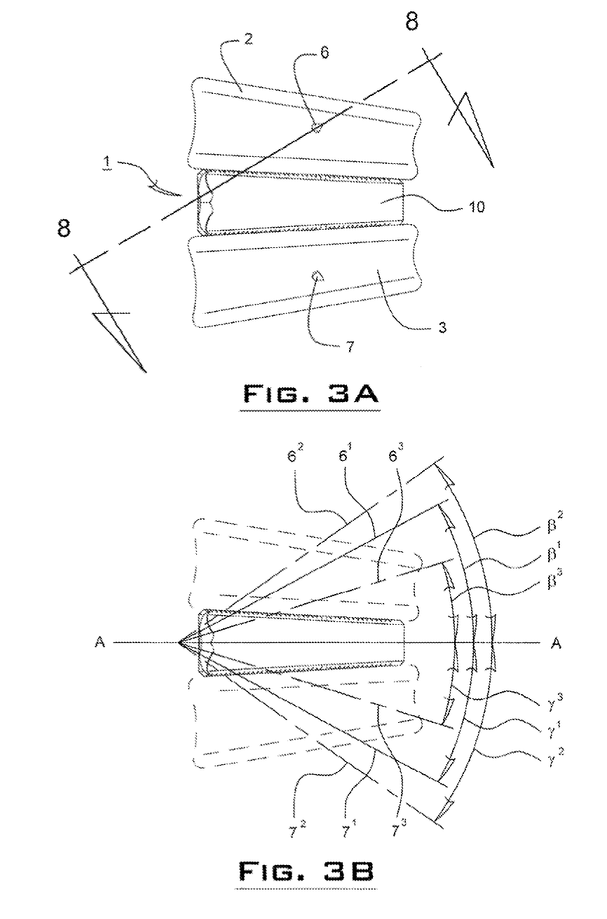

Referring now to FIG. 3A, a lateral environmental view of the fusion device 1 of the present invention is shown disposed in the disc space S between the superior vertebral body 2 and the inferior vertebral body 3, showing a section cut 8-8 through an oblique plane along vertebral screws 21 and 25. The right superior bi-cortical purchase 6 and right inferior bi-cortical purchase 7 obtained by vertebral screws 25 and 26 may be more clearly seen. An advantage of the present invention is the device 1 provides for bi-cortical purchase of a vertebral body.

Referring now to FIG. 3B, a lateral view of the fusion device 1 of FIG. 3A is shown disposed in the disc space between a superior vertebra and a inferior vertebra, shown in phantom, revealing bi-cortical purchase axes 6¹, 6² and 6³ that may be achieved by an ascending guide and bi-cortical purchase axes 7¹, 7² and 7³ that may be achieved by a descending guide. Each guide has an attitude, defined by an axis composed of a vertical component and horizontal component, which directs a vertebral screw toward a desired target location for penetration into the vertebra. The bi-cortical purchase axes 6¹, 6² and 6³ each have a corresponding angle β¹, β² and β³ in the vertical plane with respect to the horizontal plane identified by A-A. Likewise, the bi-cortical purchase axes 7¹, 7² and 7³ have a corresponding angle γ¹, γ² and γ³ in the vertical plane with respect to the horizontal plane identified by A-A. Angles in the horizontal plane are not shown; however such angles would be measured with respect to a sagittal plane.

Those skilled in the art will immediately recognize that factors such as vertebra type (cervical, thoracic or lumbar) and vertebral height as well as and height of the disc space S may affect the desired attitude of a guide. For example, a desired attitude to achieve a right superior bi-cortical purchase may be 31.87 degrees in the vertical plane and 34.17 degrees in the horizontal plane.

Referring now to FIG. 4A, a perspective view of the fusion device 1 of the present invention is shown, showing a section line 4B-4B cut through the midsagittal plane, bisecting an ascending guide 43 and descending guide 44. The cage 10 is annular in shape, and as used herein, the term “annular” shall be defined to mean ring-like, where the shape of the element described ranges from circular to that of the contour of the body of a vertebra, kidney shaped, rectangular or rectangular with rounded corners.

The cage 10 has a wall 11 with a thickness defined by an outer surface 12 and an inner surface 14. In the preferred embodiment, the outer surface 12 follows the contour of the body of a vertebra. The inner surface 14 is defined by a cavity 13 within the cage 10. The cage 10 has a top surface 16 and a bottom surface 18 that are in a spatial relationship to one another. The cage 10 of the present embodiment has a plurality of ascending guides 41, 43, 45 extending through the wall 11 from the outer surface 12 to the top surface 16. A plurality of descending guides likewise extend through the wall 11 from the outer surface 12 to the bottom surface 18.

Although a plurality of guides are shown in the present embodiment, it is the intent of the present invention to provide at least one ascending guide extending through the wall 11 from the outer surface 12 through the top surface 16 and at least one descending guide extending through the wall 11 from the outer surface 12 through the bottom surface 18.

The top surface 16 and bottom surface 18 preferably have denticulations 17 to provide a bite on the superior vertebral body 2 and the inferior vertebral body 3, respectively, to prevent slipping. In one embodiment, the outer surface of the cage has a shape that generally follows the contour of the body of a vertebra.

Referring now also to FIG. 4B a section cut of 4B-4B of FIG. 4A through the midsagittal plane of the cage 10, bisecting the ascending guide 43 and descending guide 44 is shown. Each guide 43, 44 has an axis 63, 64 defined by an attitude that directs a vertebral screw toward a desired target for penetration into the vertebra. Each of the guides 43, 44 have a corresponding seat 53, 54. In the preferred embodiment, each guide has a corresponding seat to provide a surface for the head of the vertebral screw to rest or shoulder against. Additionally, each seat may be recessed and have a hemispherical contour, or a ball and socket relationship with a vertebral screw.

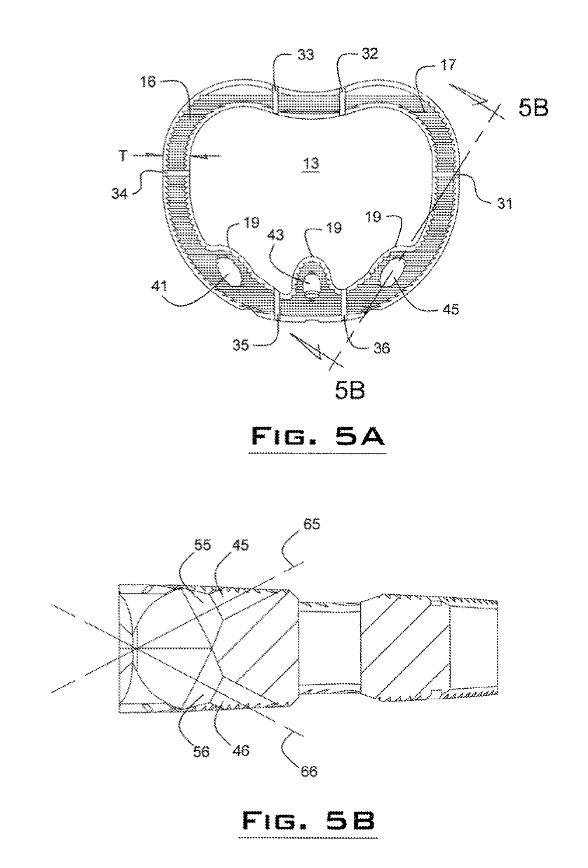

Referring now to FIG. 5A, a top view of the fusion device 1 of the present invention is shown. The wall 11 of the cage 10 has a thickness T that may be relatively uniform or variable. A plurality of bosses 19 are provided to add additional strength to the ascending guides 41, 43, and 45. A plurality of raidially extending grooves 31, 32, 33, 34, 35, 36 are disposed on the top surface 16 and bottom surface 18 of the cage 10. The grooves 31-36 are adapted to retain sutures (not shown) that span the cavity 13 to retain graft material (not shown) in the cavity 13.

Referring now to FIG. 5B, a section cut along 5B-5B of the device of FIG. 5A through an oblique plane reveals a cross section of ascending guide 45 and descending guide 46. Each guide 45, 46 has a corresponding axis 65, 66 defined by an attitude that directs a vertebral screw in a desired trajectory. In the preferred embodiment, the trajectory for a vertebral screw directed by guides 45 and 46 will result in a bi-cortical purchase of a superior and inferior vertebra, respectively. The guides 41-46 of the exemplary embodiment have a circular cross section.

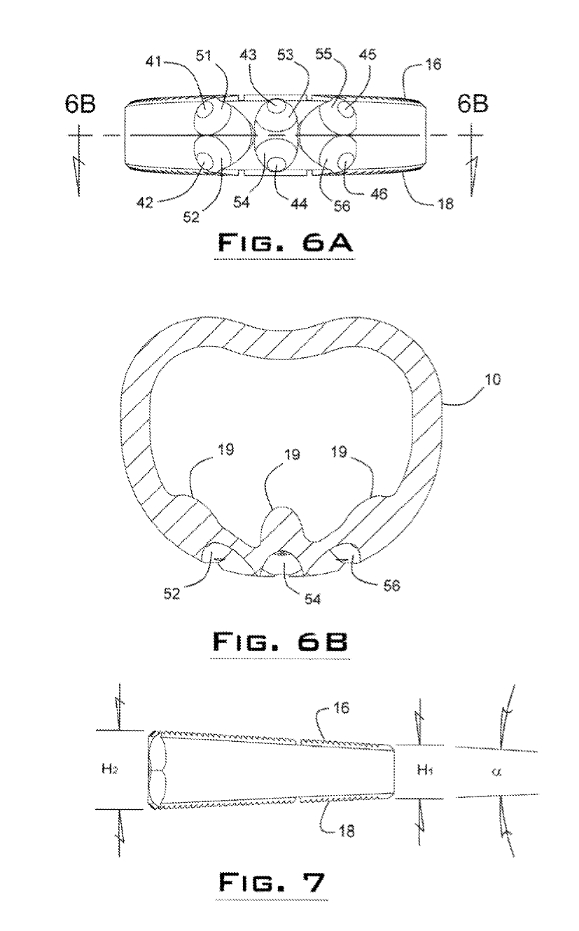

Referring now also to FIG. 6A, an anterior view of the fusion device 1 of the present invention is shown revealing six screw guides 41-46 and six seats 51-56, where each seat cooperates with a guide. In the present embodiment, material is scalloped out of the cage 10 to provide access to vertebral screws (not shown) shouldered against the seats 51-56. The cage 10 has three ascending screw guides 41, 43, and 45 and three descending screw guides 42, 44, and 46. Guides 41, 43, and 45 are referred to as ascending guides because their function is to direct vertebral screws in an upward direction from the outer surface 12 through the top surface 16. Likewise, guides 43, 44, and 46 are referred to as descending screw guides because they direct vertebral screws in a downward direction from the outer surface 12 through the bottom surface 18.

The ascending guides 41, and 45 are disposed to permit bi-cortical screw purchase of a superior vertebra and said descending guides 42, and 46 are disposed to permit a bi-cortical screw purchase of an inferior vertebra. The ascending guide 43 is disposed to permit cancellous screw purchase of a superior vertebra 2. The descending guide 44 is disposed to permit cancellous screw purchase of the inferior vertebra 3.

Referring now to FIG. 6B, a cross section cut along 6B-6B of the cage 10 of FIG. 6A through a horizontal plane is shown to reveal an inferior half of the cage 10. In the exemplary embodiment, the descending guides 42, 44, 46 are located in the inferior half of the cage and the ascending guides 41, 43, 45 are located in the superior half of the cage. However, it should be understood by those skilled in the art that ascending guides may be disposed in the inferior half of a cage and descending guides may be disposed in the superior half of the cage.

Referring now to FIG. 7, a lateral view of the fusion device 1 of FIG. 4A is shown, revealing a Lordosis angle α between the top surface 16 and bottom surface 18. The spatial relationship of the top surface 16 of the cage 10 and bottom surface 18 of the cage 18 is at least partially defined by the Lordosis angle. The anterior side of the cage 10 has a height H₂ and the posterior end of the cage has a height H₁. The top surface 16 may lie within a first plane and the bottom surface 18 may lie within a second plane. It is anticipated that the cage 10 may be manufactured in various sizes, including various heights H₁ and H₂ as well as various Lordosis angles.

Referring now to FIG. 8, a section cut along 8-8 of the fusion device of FIG. 3 through an oblique plane bisecting vertebral screws 21 and 25 is shown. The screws 21, 25 are within the harder, stronger cortical bone 5 of the superior vertebrae 2. The ascending guides in the present embodiment are disposed at oblique angles to permit bi-cortical screw purchase 6, 8 of the superior vertebra 2. Although not shown, the descending guides in the present embodiment are disposed at oblique angles to permit bi-cortical screw purchase 7, 9 of the inferior vertebra 3.

Referring now to FIG. 9 a section cut along 9-9 through a midsagittal plane is shown bisecting cancellous vertebral screws 23 and 24 of the fusion device of FIG. 2. The guides disposed along the midsagittal plane of the cage 10 direct vertebral screws 23 and 24 into a cancellous purchase of the superior vertebra 2 and inferior vertebra 3. The screws 23 and 24 are in contact with the seats 53 and 54 respectively. The seats 53, 54 aid in distributing the force of the screws across a wider area. The cage 10 is disposed between a superior vertebra 2 and inferior vertebra 3, where the top surface 16 of the cage 10 is in contact with the vertebral body 2 of the superior vertebra and the bottom surface of the cage 10 is in contact with the vertebral body 3 of the inferior vertebra.

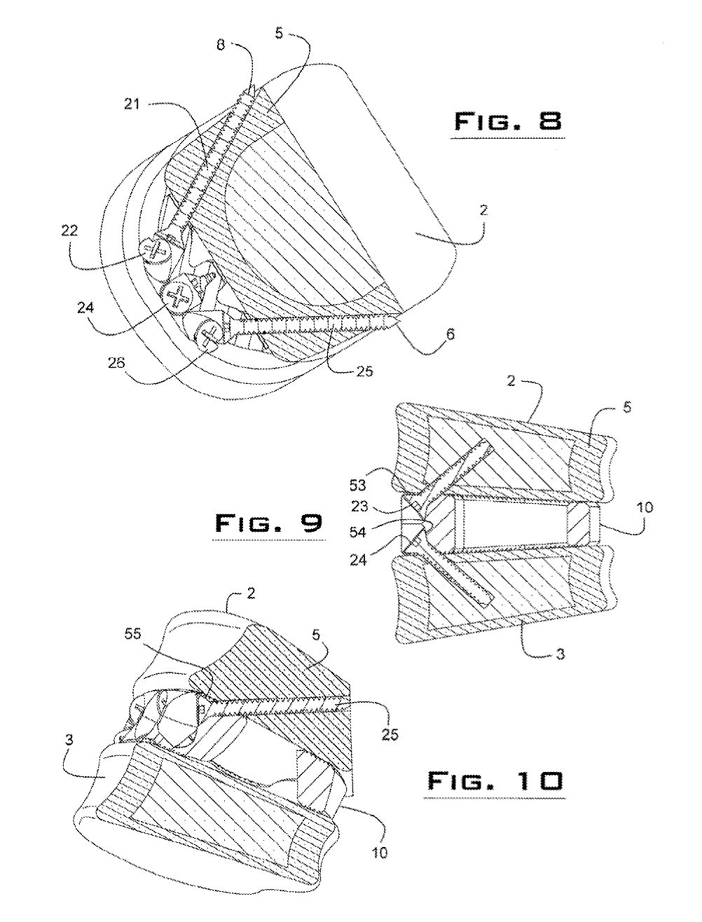

Referring now to FIG. 10, a section cut along 10-10 of the fusion device of FIG. 1, provides a view of an oblique plane'bisecting vertebral screw 25. As may be seen, the cortical vertebral screw 25 is directed at an oblique angle by the guide 45 (not shown in this figure) which is disposed at an oblique angle within the cage 10 to permit bi-cortical purchase of the cortical bone 5. The screw 25 is in contact with the seat 55 for force distribution.

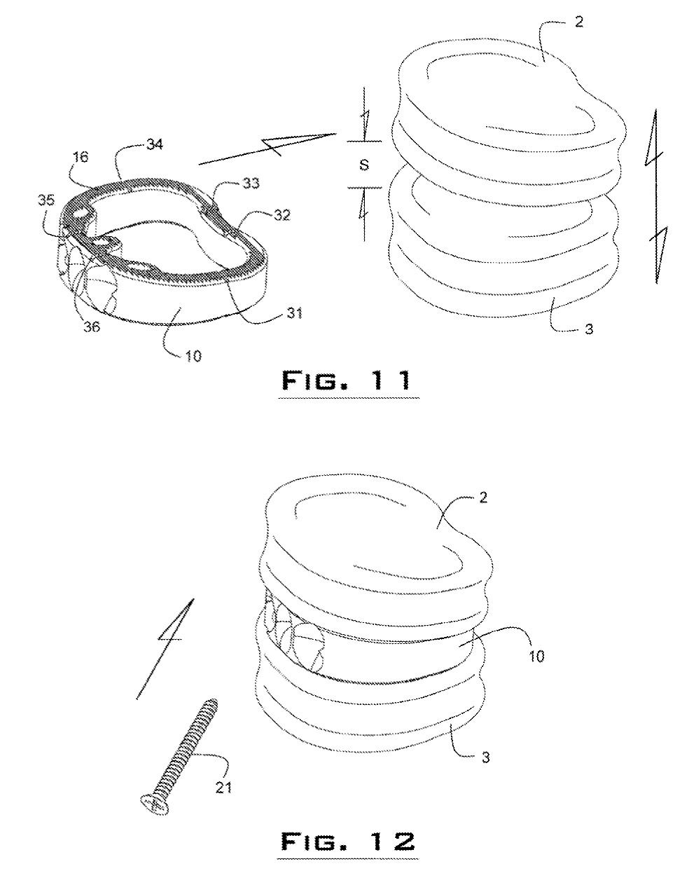

Referring now to FIG. 11, a perspective view of the fusion device 1 of the present invention revealing the method for inserting the device 1 into the disc space S between the superior vertebra 2 and inferior vertebra 3 is shown. Once a discectomy is completed, the space S between the adjacent vertebrae 2, 3 formerly occupied by the intervertebral disc is ready to receive the stand alone anterior fusion device 1 of the present invention.

Referring now to FIG. 12 a perspective environmental view of the fusion device 1 of the present invention is shown disposed between the superior vertebra 2 and inferior vertebra 3, revealing the direction vertebral screw 21 will be inserted into the cage 10, as governed by an ascending screw guide 41 from FIG. 6. In practice, the cage 10 is disposed between the superior vertebra 2 and inferior vertebra 3, where the top surface 16 of the cage 10 is in contact with the end plate (not shown) of the superior vertebra 2 and the bottom surface 18 of the cage 10 is in contact with the end plate (not shown) of the inferior vertebra 3. However, for purposes of the present invention, the top surface 16 of the cage 10 is adjacent to the vertebral body of the superior vertebra 2 and the bottom surface 18 of the cage 10 is adjacent to the vertebral body of the inferior vertebra 3. It is preferred that at least one ascending guide, for example 41 from FIG. 6, is disposed to permit bi-cortical screw purchase of a superior vertebra 2 and at least one descending guide, for example 42 from FIG. 6, is disposed to permit a bi-cortical screw purchase of an inferior vertebra 3. The ascending guides 41, 45 are disposed at oblique angles to permit bi-cortical screw purchase of the superior vertebra 2 and descending guides 42, 46 may be disposed at oblique angles to permit bi-cortical screw purchase of the inferior vertebra 3. Additionally, ascending guide 43 and descending guide 44 are disposed to provide cancellous screw of the superior vertebra 2 and inferior vertebra 3, respectively.

Referring now to FIG. 13, a perspective view of the fusion device 1 of the present invention is shown packed with graft material 15. Grooves 31-36 are raidially disposed on the top surface 16 and bottom surface 18 of the cage 10 are adapted to retain sutures 37, 38, 39 that span the cavity 13 to retain graft material 15 in the cavity 13 of the cage 10.

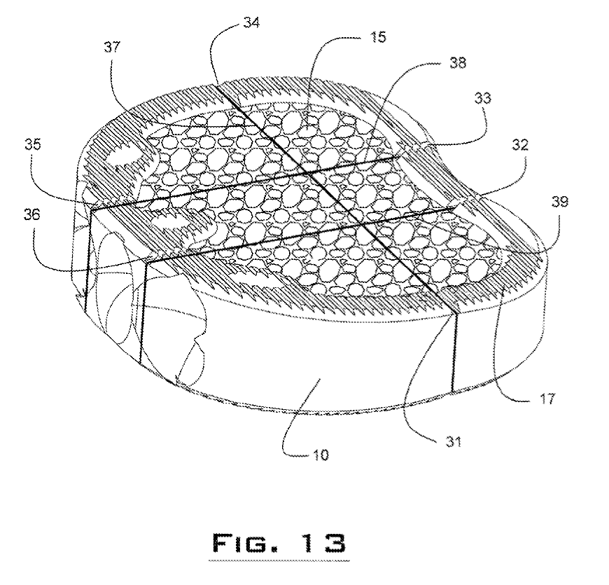

Referring now to FIG. 14, a perspective environmental view of an alternate embodiment of the fusion device 101 according to the principles of the present invention is shown. In the exemplary embodiment, a four screw symmetrically distributed device 101 is disposed in the disc space between a superior vertebra 102 and an inferior vertebra 103, showing section line 17-17 cutting through an oblique plane bisecting vertebral screws 122 and 124. A right superior bi-cortical purchase 106 and right inferior bi-cortical purchase 107 are obtained by vertebral screws 120 of sufficient length and angle as directed by guides (not shown) disposed within the cage 110.

Referring now to FIG. 15, an anterior environmental view of an alternate embodiment of the fusion device 101 of the present invention is shown disposed in the disc space between a superior vertebra 102 and a inferior vertebra 103. Four screws, 121, 122, 123 and 124 are disposed in their respective guides (not shown in this figure), whereby right superior bi-cortical purchase 106 and right inferior bi-cortical purchase 107 is obtained by vertebral screws 122 and 124 and left superior bi-cortical purchase 108 and left inferior bi-cortical purchase 109 is obtained by vertebral screws 121, 123.

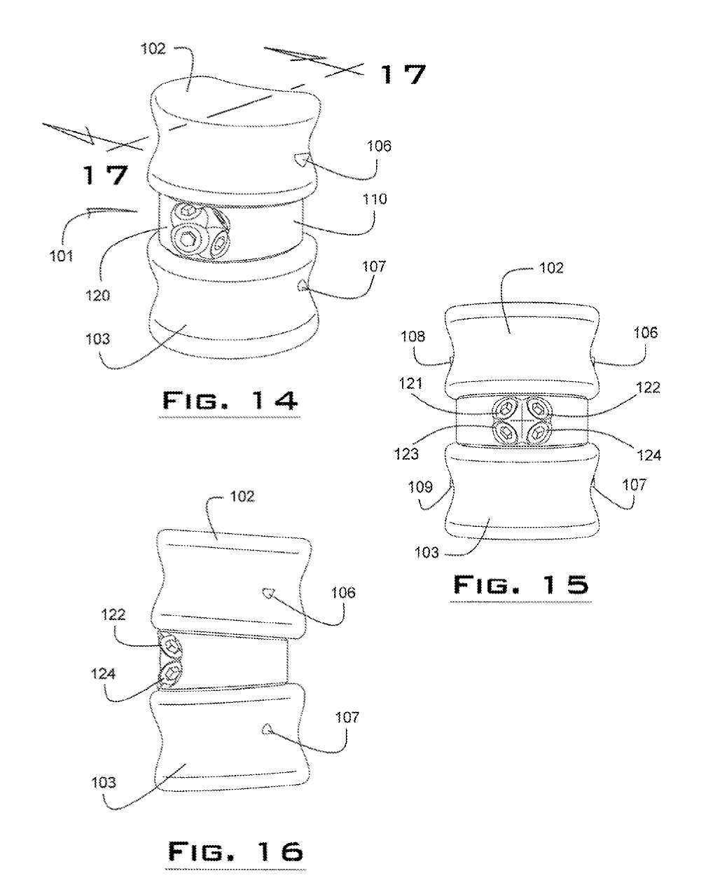

Referring now to FIG. 16, a lateral environmental view of an alternate embodiment of the fusion device of the present invention is shown disposed in the disc space between a superior vertebra 102 and an inferior vertebra 103. A right superior bi-cortical purchase 106 and right inferior bi-cortical purchase 107 are obtained by vertebral screws 122 and 124.

Referring now to FIG. 17, an oblique sectional cut through the device 101 and vertebral bodies 102, 103 of FIG. 14 reveals a cross section of ascending screw 122 and descending screw 124. The guides (not shown in this figure) may be formed with a diameter sufficient to provide clearance between each guide and the respective vertebral screws 121, 122, 123, 124 to permit a change in angulation of vertebral screws 121-124. As used herein, angulation shall refer to the angle of the vertebral screw with respect to one or more reference planes of the cage, such as a horizontal plane, sagittal plane, top surface (not shown) or bottom surface (not shown) of the cage 110. The angle β reveals the available angulation of the screw 122. To promote a change in angulation, each seat may be conical or hemispherical to permit angulation about the axis of a guide.

Although it is preferred that the present embodiment of device 101 permit change in angulation is provided by all guides, it should be understood that at least one ascending guide (not shown) has a diameter sufficient to provide a change in angulation of a vertebral screw 122 to permit bi-cortical screw purchase of a superior vertebra 102 and at least one descending guide (not shown) has a diameter sufficient to provide change in angulation of a vertebral screw 124 to permit bi-cortical screw purchase of an inferior vertebra.

Referring now to FIG. 18, a perspective environmental view of an alternate embodiment of the fusion device 101 of the present invention is shown with the superior vertebra 102 removed and a bi-cortical screw purchase 107 of the inferior vertebra 103. Although not shown in this figure, the ascending guides are disposed at oblique angles to direct the vertebral screws toward a bi-cortical screw purchase of the superior vertebra, as illustrated by vertebral screws 121 and 122. Likewise, the descending guides are also disposed at oblique angles to direct vertebral screws toward a bi-cortical purchase of the inferior vertebra.

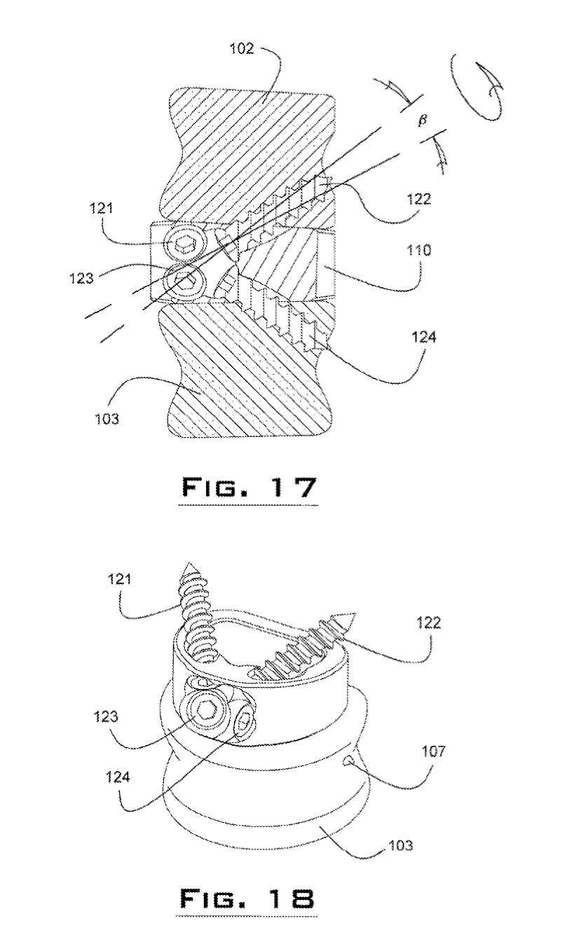

Referring now to FIG. 19, a perspective view of an alternate embodiment of the fusion device 101 of the present invention is shown. An outer surface 112 and inner surface 114 define the wall thickness. In the present embodiment, the wall thickness is relatively uniform. At least one descending guide 143 is shown. Seats 153 and 154 provide surface area for force distribution of the tension on the vertebral screws A top surface 116 is free of denticulations and suture grooves.

Referring now to FIG. 20, a top view of an alternate embodiment of the fusion device 101 of the present invention is shown. The shape of the guide holes 141, 142 exiting the top surface 116 suggest an oblique angle that is close to being within the plane of the top surface 116. A cavity 113 is provided to receive graft material.

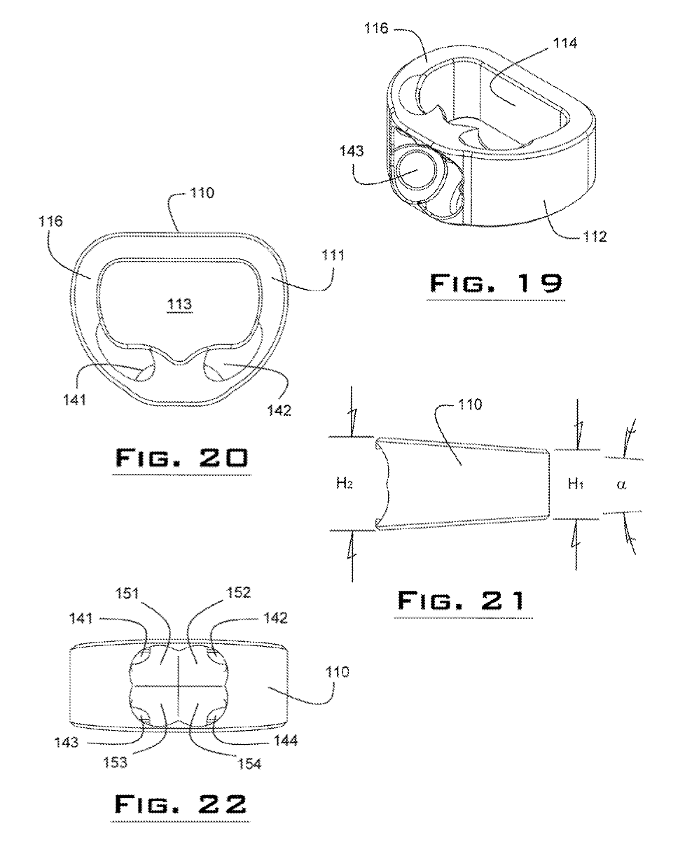

Referring now to FIG. 21, a lateral view of an alternate embodiment of the fusion device of the present invention is shown, revealing a Lordosis angle α. The Lordosis angle reflects the angle formed by the bottom surface of the superior vertebral body and the top surface of the inferior vertebral body when in a proper special relationship. A properly sized cage 110 will maintain the adjacent vertebra in proper alignment and after the cortical vertebral screws are installed, the superior and inferior vertebra will be stabilized.

Referring now to FIG. 22, an alternate embodiment of the fusion device 101 of the present invention is shown, revealing four screw guides and four seats. The cage 110 of the present embodiment provides four guides, two ascending and two descending. The ascending guides 141, 142 and descending guides 143, 144 are disposed symmetrically with respect to the midsagittal plane of the cage 110.

Referring now to FIG. 23, a perspective environmental view of another alternate embodiment of the fusion device 201 according to the principles of the present invention is shown. In the exemplary embodiment, a four screw non-symmetrical device 201 is disposed in the disc space between a superior vertebra 202 and a inferior vertebra 203, showing section line 26-26 cutting through a sagittal plane and bisecting vertebral screw 223 and line 28-28 cutting through an oblique plane and bisecting screw 224. A right superior bi-cortical purchase 206 and right inferior bi-cortical purchase 207 are obtained by vertebral screws 220 of sufficient length and angle as directed by guides (not shown) disposed within the cage 210.

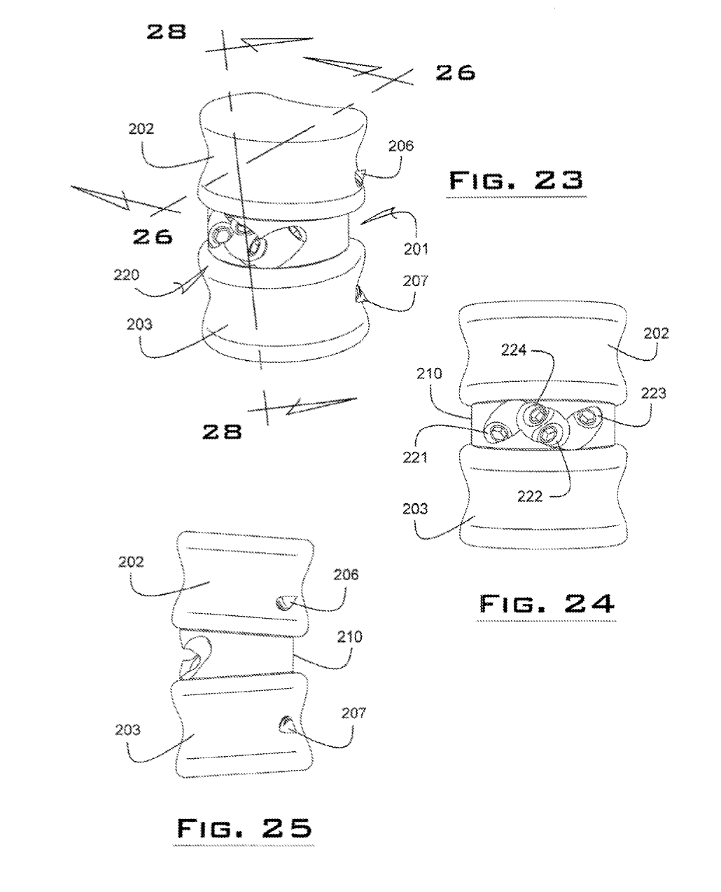

Referring now to FIG. 24, an anterior environmental view of another alternate embodiment of the fusion device 201 of the present invention is shown disposed in the disc space between a superior vertebra 202 and an inferior vertebra 203. Four screws, 221, 222, 223 and 224 are disposed in their respective guides (not shown in this figure). In the present embodiment, the guides are disposed asymmetrically with respect to the midsagittal plane of the cage 210.

Referring now to FIG. 25, a lateral environmental view of another alternate embodiment of the fusion device 201 of the present invention disposed is shown in the disc space between a superior vertebra and a inferior vertebra. A right superior bi-cortical purchase 206 and right inferior bi-cortical purchase 207 are obtained by vertebral screws 223 and 222, respectively.

Referring now to FIG. 26, a sectional cut along line 26-26 of the device 201 and vertebral bodies 202, 203 of FIG. 23 is shown through a sagittal plane to reveal a cross section of ascending screw 223. In this embodiment, the point of purchase of the screw 223 in the cortical bone 205 is below the midpoint of the hourglass shape of the body of the superior vertebrae 202.

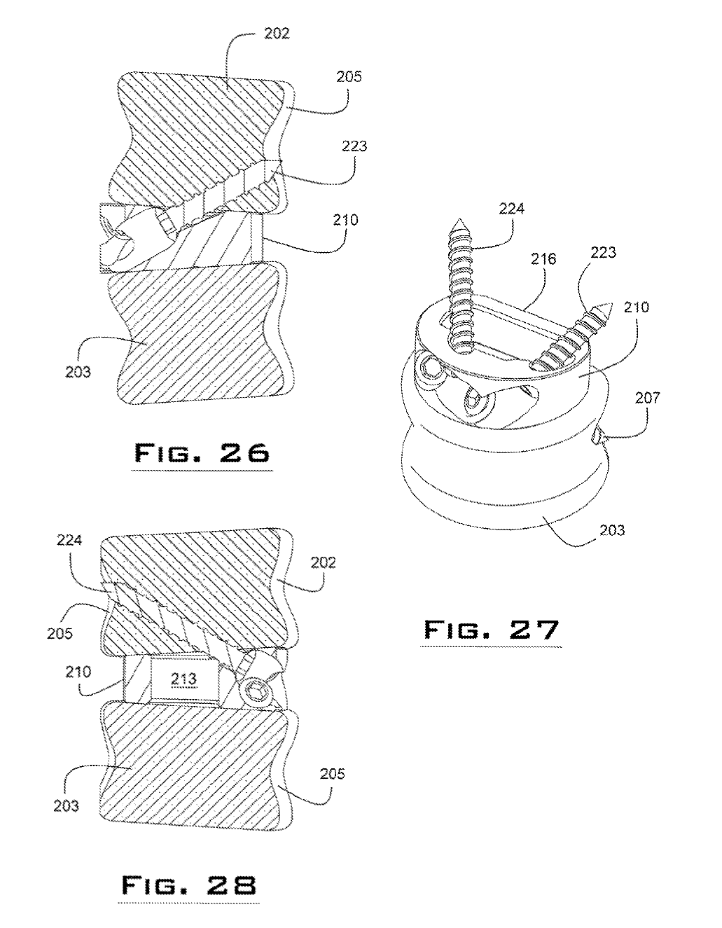

Referring now to FIG. 27, a perspective environmental view of another alternate embodiment of the fusion device 201 of the present invention is shown with the superior vertebra removed and a bi-cortical screw purchase 207 of the inferior vertebra 203. With reference further to FIG. 28, the angle of the vertebral screws 224, 223 reveal that each of the ascending guides has a different slope with respect to a first plane, wherein the top surface 216 lies within the first plane. Although not shown, each of the descending guides has a different slope with respect to a second plane, wherein the bottom surface of said cage 210 lies within the second plane.

Referring now to FIG. 28, a sectional cut along line 28-28 of the device 201 and vertebral bodies 202, 203 of FIG. 23 is shown through an oblique plane to reveal a cross section of ascending screw 224. In this embodiment, the point of purchase of the screw 224 in the cortical bone 205 is above the midpoint of the hourglass shape of the body of the superior vertebrae 202.

Referring now to FIG. 29, a perspective view of another alternate embodiment of the fusion device 201 of the present invention is shown. The seats are shown recessed into the wall 211. The guides penetrating through the top surface 216 of the cage 210 reveal the ascending guides penetrate the top surface 216 of the cage 210 at different angles. Referring now also to FIG. 30, a top view of another alternate embodiment of the fusion device 201 of the present invention is shown. The outer surface 212 and inner surface 214 form the thickness of the wall 211. The interior surface 214 is defined by the cavity 213. A first and second ascending guides 243, 244 are shown projecting through the top surface 216 of the cage 210.

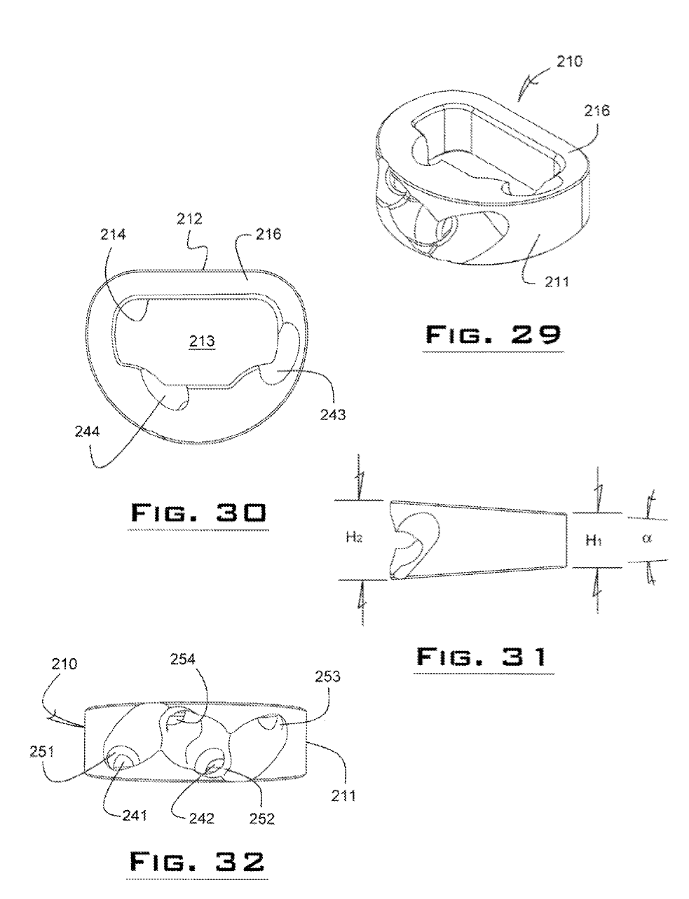

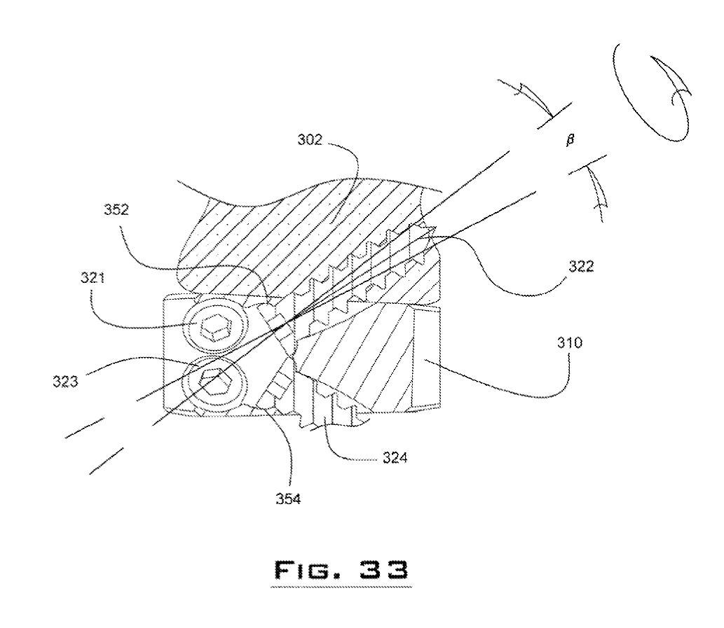

Referring now to FIG. 31, a lateral view of another alternate embodiment of the fusion device 201 of the present invention is shown revealing a Lordosis angle α and anterior and posterior heights H₁ and H₂. In practice, the surgeon measures the disc space after removal of the intervertebral disc and identifies the correct size of the anterior fusion device 201 of the present invention to fit into the disc space. Measurements may include α, H₁ and H₂.

Referring now to FIG. 32, an anterior view of another alternate embodiment of the fusion device of the present invention is shown, revealing four screw guides 241-244 and four seats 251-254. In the present embodiment, the ascending guides 243, 244 and descending guides 241, 242 are disposed asymmetrically with respect to the midsagittal plane of the cage 210.

Referring now to FIG. 33, an alternate embodiment of the device 101 of FIG. 17 is shown having hemispherical seats 352 and 354 and corresponding hemispherical headed ascending cortical screw 322 and hemispherical headed descending cortical screw 324. Cortical screws 321 and 323 also have a spherical head to permit a ball and socket engagement with their respective seats. Angle β reveals the change in angulation permitted by the guides having a diameter sufficient to provide clearance to the screws 321-324 to allow the screws 321-324 to have a trajectory that is non collinear with the axis of each guide with the cooperation of the hemispherical seats. Once the screws 321-324 are inserted, they are fixed and fix the cage 310 with respect to the adjacent vertebrae. The cortical screw 322 is directed by a guide to achieve bi-cortical purchase of a superior vertebra 302.

### Example

An example of the method for stabilizing adjacent vertebrae through an anterior approach by inserting the stand-alone device 1 of the present invention between the vertebrae to be fused will now be described.

A surgeon provides an anterior approach to a spine where the intervertebral disc to be removed is located and readies the patient for the anterior fusion device. The spine surgeon removes the failed intervertebral disc through the anterior approach. The spine surgeon provides an anterior fusion device comprising an annular cage having a wall with a thickness defined by an outer surface and an inner surface. The inner surface of the device is defined by a cavity therein. The cage has a top surface and a bottom surface, where the top surface is in a spatial relationship to the bottom surface. The cage has at, least one ascending guide extending through the wall from the outer surface through the top surface and at least one descending guide extending through the wall from the outer surface through the bottom surface. The cavity is packed with bone graft. In the preferred method, the bone graft is secured to the cage with sutures, where the sutures are at least partially retained by grooves formed in the cage. The anterior fusion device is inserted into the disc space between the adjacent vertebrae. A vertebral screw is inserted into each screw guide, whereby the angle of the screw guide directs the screw to obtain a bi-cortical purchase.

The foregoing discussion discloses and describes the preferred structure and control system for the present invention. However, one skilled in the art will readily recognize from such discussion, and from the accompanying drawings and claims, that various changes, modifications and variations can be made therein without departing from the true spirit and fair scope of the invention as defined in the following claims.

What is claimed is:

1. A fusion device for providing a desired spatial relationship between adjacent vertebrae, comprising:
an annular cage having a wall with a thickness defined by an outer surface and an inner surface, said inner surface defined by a cavity therein, said cage having a top surface and a bottom surface, said top surface being in a spatial relationship to said bottom surface, said cage having at least one ascending guide extending through said wall from said outer surface through said top surface and at least one descending guide extending through said wall from said outer surface through said bottom surface, each said guide having an axis defined by an attitude, whereby said guide directs a vertebral screw in a desired trajectory.

2. The device of claim 1, further comprising a second ascending guide extending through said wall from said outer surface through said top surface and a second descending guide extending through said wall from said outer surface through said bottom surface.

3. The device of claim 2, wherein said top surface lies within a first plane and said bottom surface lies within a second plane.

4. The device of claim 3, wherein each of said ascending guides has a different slope with respect to the first plane.

5. The device of claim 3, wherein each of said descending guides has a different slope with respect to the second plane.

6. The device of claim 1, wherein each guide has a seat.

7. The device of claim 1, wherein said top surface and said bottom surface are denticulated.

8. The device of claim 1, wherein said outer surface has a shape that generally follows the contour of the body of a vertebra.

9. The device of claim 6, wherein each seat is recessed.

10. The device of claim 1, further comprising a cortical vertebral screw disposed in said ascending guide and a cortical vertebral screw disposed in said descending guide.

11. The device of claim 2, wherein said ascending guides and said descending guides are disposed symmetrically with respect to the midsagittal plane of said cage.

12. The device of claim 2, wherein said ascending guides and said descending guides are disposed asymmetrically with respect to the midsagittal plane of said cage.

13. The device of claim 1, wherein said cage is disposed between a superior vertebra and inferior vertebra, said top surface of said cage is adjacent to the vertebral body of the superior vertebra and said bottom surface of said cage is adjacent to the vertebral body of the inferior vertebra, said at least one ascending guide is disposed to permit bi-cortical screw purchase of a superior vertebra and said at least one descending guide are disposed to permit a bi-cortical screw purchase of an inferior vertebra.

14. The device of claim 2, wherein said cage is disposed between a superior vertebra and inferior vertebra, said top surface of said cage is adjacent to the vertebral body of the superior vertebra and said bottom surface of said cage is adjacent to the vertebral body of the inferior vertebra, said ascending guides are disposed to permit bi-cortical screw purchase of a superior vertebra and said descending guides are disposed to permit a bi-cortical screw purchase of an inferior vertebra.

15. The device of claim 14, wherein said ascending guides are disposed at oblique angles to permit bi-cortical screw purchase of the superior vertebra and said descending guides are disposed at oblique angles to permit bi-cortical screw purchase of the inferior vertebra.

16. The device of claim 6, wherein said at least one ascending guide has a diameter sufficient to provide a change in angulation of a vertebral screw to permit bi-cortical screw purchase of a superior vertebra and said at least one descending guide has a diameter sufficient to provide a change in angulation of a vertebral screw to permit bi-cortical screw purchase of an inferior vertebra.

17. The device of claim 1, wherein the spatial relationship of said top surface of said cage and said bottom surface of said cage is at least partially defined by a Lordosis angle.

18. The device of claim 1, further comprising a plurality of grooves raidially disposed on the top surface and bottom surface of said cage, said grooves adapted to retain sutures that span said cavity to retain graft material in said cavity.

19. A fusion device for providing a desired spatial relationship between adjacent vertebrae, comprising:
an annular cage having a wall with a thickness defined by an outer surface and an inner surface, said inner surface defined by a cavity therein, said cage having a top surface and a bottom surface, said top surface being in a spatial relationship to said bottom surface, said cage having a plurality of ascending guides extending through said wall from said outer surface through said top surface and a plurality of descending guides extending through said wall from said outer surface through said bottom surface.

20. The fusion device of claim 19, wherein said cage is disposed between a superior vertebra and inferior vertebra, said top surface of said cage being in contact with the vertebral body of the superior vertebra and said bottom surface of said cage being in contact with the vertebral body of the inferior vertebra, said ascending guides are disposed to permit bi-cortical screw purchase of a superior vertebra and said descending guides are disposed to permit a bi-cortical screw purchase of an inferior vertebra.

21. A method for stabilizing adjacent vertebrae by using an anterior fusion device, the method comprising:
providing an anterior approach to a spine;
providing an anterior fusion device comprising a an annular cage having a wall with a thickness defined by an outer surface and an inner surface, the inner surface defined by a cavity therein, the cage having a top surface and a bottom surface, the top surface being in a spatial relationship to the bottom surface, the cage having at least one ascending guide extending through the wall from the outer surface through the top surface and at least one descending guide extending through the wall from the outer surface through the bottom surface, each guide having an axis defined by an attitude, whereby each guide directs a vertebral screw in a desired trajectory;
packing the cavity with bone graft;
inserting the anterior fusion device into the disc space between adjacent vertebrae; and
inserting a vertebral screw into each screw guide, whereby the screw guide permits bi-cortical screw purchase.

22. The method for stabilizing adjacent vertebrae by using an anterior fusion device as set forth in claim 21, further comprising the step of:
securing the bone graft to the cage with sutures, the sutures being at least partially retained by grooves formed in the cage.
